(19) United States
(12) Patent Application Publication
(10) Pub. No.: US 2012/0295342 A1
(43) Pub. Date: Nov. 22, 2012

(54) PREVENTION OF CONTAMINATION OF FEED RESERVOIRS & FEED LINES IN BIOREACTOR SYSTEMS

(75) Inventor: Robert D. FALLON, Elkton, MD (US)

(73) Assignee: E. I. DU PONT DE NEMOURS AND COMPANY, Wilmington, DE (US)

(21) Appl. No.: 13/297,514

(22) Filed: Nov. 16, 2011

Related U.S. Application Data
(60) Provisional application No. 61/414,916, filed on Nov. 18, 2010.

(51) Int. Cl. C12M 1/12; A61L 2/04

(52) U.S. Cl. 435/289.1; 422/22

(57) ABSTRACT

A method to prevent contamination of feed line(s) and nutrient feed reservoir(s) is disclosed. In this method a heated zone is established in the feed line close to the location where the feed line connects to the bioreactor. The heated zone prevents back growth of cells from the bioreactor back into the feed line and further into the feed reservoir.

This application claims the benefit of U.S. Provisional Application 61/414,916, filed Nov. 18, 2010, and is incorporated by reference in its entirety.

### FIELD OF THE INVENTION

The current method relates to the field of microbiology and contamination prevention in bioreactor systems. Specifically, it relates to a method for prevention of contamination of feed reservoirs and feed lines providing nutrients to bioreactors.

### BACKGROUND OF THE INVENTION

Bioreactor systems are commonly used for growth and production of microbial, mammalian and plant cells for various industrial and pharmacological applications. One of the universal problems in operating bioreactor systems is the contamination problem which results in process failure. Contamination prevention in bioreactor systems is therefore important to allow uninterrupted production of the desired cells and/or products and to meet manufacturing and production timelines. Additionally, interruption of contaminated bioreactors, clean ups and start ups are costly and can have significant economical impact on the entire process.

Various methods have been used to prevent contamination or the spread thereof. Some of these methods depend on application of technologies detrimental to cell growth such as using lethal temperatures, ultraviolet radiation, adding chemical inhibitors to the growth medium and using ionizing radiations. For example:

U.S. Pat. No. 4,192,988 disclosed application of electrical heating for heating a sink drain barrier to prevent growth of microorganisms in the drain.

U.S. Pat. No. 3,985,994 disclosed application of an electric heater to heat the interior of the connecting pipe portion between the outlet pipe and the drain pipe of a wash basin to prevent microbes from rising from the drain pipe into the outlet pipe.

Application of ultraviolet light for contamination control in blood products was disclosed in WO01174407. U.S. Patent Publication 2008/0142452 disclosed use of UV light in killing microorganisms during water treatment.

Application of intervening physical devices such as air-breaks and filters that physically interrupt penetration of cells and thus prevent contamination has been known and commonly practiced by those knowledgeable in the art (Stanbury, P. R. et al., Principles of Fermentation Technology, 2^{nd} Edition. 1995, Elsevier Sciences Limited, Burlington, Mass.). In the commonly owned application publication WO 2004101479 filtration was used to remove microbial cells from the product stream of the bioreactor.

Application of bioreactor systems for production of various industrial chemicals and pharmaceutical products has been increasing over the past two decades, thus there is a need for developing effective and economical methods to prevent contamination in these systems without application of intervening physical devices and/or harsh chemicals.

### SUMMARY OF THE INVENTION

The method disclosed herein addresses the need for preventing contamination in bioreactor systems. Specifically, the method teaches prevention of contamination of the feed reservoir and the feed line in a bioreactor system during its operation. In the currently disclosed method, no intervening physical device in either the feed reservoir or the feed line is used for contamination prevention. Rather contamination prevention is effected through establishing a heated zone in the feed line wherein the heated zone prevents penetration and back growth of cells from the bioreactor into the feed line and consequently prevents contamination of the feed reservoir.

In an aspect, the present invention is a method for preventing contamination in a bioreactor system comprising applying heat to a feed line supplying a growth medium from a feed reservoir to the bioreactor via the feed line, said heat applied to establish a heated zone in the feed line; wherein said heated zone is sufficient to prevent contamination of the feed line or the feed reservoir with cells from the bioreactor in the absence of any intervening physical device.

In another embodiment the invention provides a bioreactor system comprising at least one reservoir containing a growth medium, a bioreactor, and at least one feed line connecting the reservoir and bioreactor having a heat source attached to at least a portion of the feed line that is a heated zone.

### BRIEF DESCRIPTION OF THE FIGURES

FIG. 1 is a graph showing the effect of heat treatment on media nutrients after various times of heating at 60° C., assessed by subsequent cell growth. Treatments are: bar 1 (vertical lines) for 6 hours, bar 2 (horizontal lines) for 12 hours, and bar 3 (white) no heat. The vertical axis is cells/ml.

FIG. 2 is a graph showing cell counts in the effluent of a bioreactor. Cell count is shown on the vertical axis and number of days is shown on the horizontal axis. The solid line shows the data for cells per milliliter in the bioreactor. Breaks in the line indicate periods when cell counts were not monitored.

### INFORMATION ON DEPOSITED STRAIN

Applicants have made the following biological deposits under the terms of the Budapest Treaty on the International Recognition of the Deposit of Microorganisms for the Purposes of Patent Procedure:

| Depositor | International | |
|---|---|---|
| Identification | Depository | Date of |
| Reference | Designation | Deposit |
| Pseudomonas stutzeri | ATCC No. PTA-11283 | Sep. 9, 2010 |
| BR5311 | | |
| Arcobacter sp 97AE3-12 | ATCC No. PTA-11409 | Oct. 14, 2010 |
| Arcobacter sp 97AE3-3 | ATCC No. PTA-11410 | Oct. 14, 2010 |
| Pseudomonas stutzeri | ATCC No. PTA-8822 | Dec. 4, 2007 |
| LH4:18 | | |
| Pseudomonas stutzeri | ATCC No. PTA-8823 | Dec. 4, 2007 |
| LH4:15 | | |

### DETAILED DESCRIPTION

This invention relates to preventing contamination during cell growth in a bioreactor. More specifically it relates to a method to inhibit back growth of cells from the bioreactor into feed line(s) from feed reservoir(s) and thus prevent contamination of the feed reservoir.

The term “bioreactor” as used herein refers to a container used to grow cells that produce one or more products, which may be the grown cells, for commercial use. The term “feed line”, as used herein, refers to the supply line that carries the feed or the growth medium from the feed reservoir to the bioreactor. The term “feed reservoir” as used herein refers to the storage container from which the growth medium or feed is drawn in order to supply the bioreactor. The term “back growth” as used herein refers to the penetration of cells from the bioreactor back into the feed line.

The present invention may be used for preventing contamination in bioreactor systems where cells of microbial, plant or mammalian origin are grown. The present invention is also particularly useful in bioreactor systems where the flow velocity of the feed is relatively low (e.g., less than 10 milliliter per minute) and run time is long (e.g., longer than 2 days). In such systems the potential for back growth of cells from the bioreactor into the feed line is increased. Flow velocity, as used herein, refers to the velocity with which the growth medium travels from the feed reservoir through the feed line to the bioreactor. Low velocity in the feed lines are common during early stages of growth in the bioreactor when cell populations are low or in late stages of a batch growth where cells growth is slow and only small amounts of feed are needed to maintain cell viability.

### Apparatus of the Current Method

A bioreactor system useful in the current method comprises one or more feed reservoir(s), a bioreactor and one or more feed lines, plus additional pipelines or tubing, one or more pumps and various control systems. The feed reservoir is connected to the bioreactor by one or more feed lines which deliver fresh growth medium from the feed reservoir to the bioreactor to support cell growth. The feed reservoir may be made of metal, glass, plastic or any other material that can be sterilized.

The bioreactor may be of any type that is commonly used in fermentation industry and is well known to those skilled in the art (Chisti, Y., Chem. Eng. Proc., 88: 80-85, 1992 and Principles of Fermentation Technology, 2^{nd} Edition. 1995, Stanbury, P. F. ed., Elsevier, Burlington, Mass.). The bioreactor can be constructed of steel or glass or any other material suitable for the specific application in mind and can be built in various sizes. The temperature of the bioreactor is maintained at the temperature optimal for the growth of the cells used in the bioreactor. The bioreactor and the feed reservoir can be connected via different pipe lines, pumps and/or tubing. One or more of these pipe lines and/or tubing can be designated the feed line that supplies fresh growth medium from the feed reservoir to the bioreactor to support cell growth. The feed line can be made of any material suitable for the specific application, which should withstand the pressure used during the process and can be sterilized. The dimensions of the feed line vary depending on the application.

The bioreactor system can have multiple feed reservoirs and multiple feed lines and can be used for a batch, a fed-batch or a continuous fermentation.

In addition, the bioreactor system may be any system where medium is fed to a population of growing cells. For example, microbial cells may be grown in a drill core sample of porous rock from an oil reservoir. The bioreactor including the drill core sample may use a core flood apparatus such as a commercial apparatus from Core Laboratories (Houston, Tex.). In this apparatus, pressure may be maintained to similar levels as encountered in subterranean oil reservoirs. New medium is fed to the microbes in the porous rock in the bioreactor through a feed line from a feed reservoir. This type of system may be used to assess the ability of the microbes to enhance oil recovery, such as by releasing oil from the porous rock sample, for use in microbial enhanced oil recovery (MEOR) processes. An example of a MEOR process is described in commonly owned and co-pending US Patent Application Publication #2011/0030956, which is herein incorporated by reference. Since the test may run for a month or more, it is desired to keep the medium from being contaminated by cells moving back up through the feed line and into the feed reservoir. The present method of establishing a heated zone in the feed line is used to control contamination of the feed reservoir, as described below.

### Temperature

Temperature is probably the most important environmental factor affecting cells' growth and viability. Since the range of temperature that permits growth of any specific cell is limited and temperatures above the maximal for growth of a particular cell may be lethal for that cell, temperature has been used as a means to prevent contamination in the art of fermentation and cell production as heat exemplified by the use of autoclaves (121° C.) for media sterilization (Stanbury, at al., supra, chapter 5). Lethal temperatures may be dependent on strain phenotype. For example cells adapted to growth at temperatures as low as 5° C. may show a lethal temperature response at 30-40° C., whereas cells adapted to growth at 95° C. may show a lethal temperature response at up to 200° C. Thus, minimum lethal temperatures can range from about 30-200° C.

In the currently disclosed method, high temperature is used to prevent back growth of the cells from the bioreactor into the feed line as described below.

For the purposes of this invention, part of the feed line between the feed reservoir and the bioreactor is established as a heated zone. The heated zone is typically from 0.1 centimeter to 100 centimeters from where the feed line joins the bioreactor. A heat source is attached to the feed line in this zone and the feed line is heated to a specific temperature. The specific medium flow rate and specific length of the heated zone determine the specific passage time through the heated zone (time of heating). “Specific passage time”, as used herein, refers to the time that the medium from the feed reservoir remains at the specified temperature in the heated zone. Specific passage time can be calculated based on heated zone volume in milliliters and flow rate in milliliters per hour. The specific temperature and specific passage time are selected for a particular cell type used in the bioreactor to prevent back growth of cells from the bioreactor by killing any cells in this zone. One skilled in the art could determine the appropriate combination of heat and specific passage time for any specific application.

In the current method the combination of specific temperature and specific passage time results in death of the cells that could have entered back from the bioreactor into the feed line and the heated zone. “Cell death” as defined herein, therefore refers to the loss of growth ability of the cells that have entered back from the bioreactor into the feed line. For the purposes of this invention it is desirable to maintain the combination of the specific passage time through the feed line in the heated zone and the temperature of the feed line in the heated zone such that during passage through the heated zone cell death is complete, while nutritional value of the growth medium is substantially retained so that the medium can support cell growth when it enters the bioreactor. “Substantially retained nutritional value” as defined herein, refers to retaining at least 90% of the initial nutritional value of the nutritional components of the growth medium after its passage through the heated zone.

In one embodiment the heated zone can have a temperature from about 30° C. to about 200° C. In another embodiment, the heated zone can have a temperature from about 40° C. to about 121° C. In one embodiment, the passage time through the heated zone can be from about 0.5 minutes to about 240 minutes. In yet another embodiment, the passage time through the heated zone can be from about 0.5 minutes to about 60 minutes. In another embodiment, the temperature in the heated zone can be from about 30° C. to about 200° C. and the specific passage time can be from about 0.5 minutes to about 240 minutes. In another embodiment, the temperature in the heated zone can be from about 40° C. to about 121° C. and the specific passage time can be from about 0.5 minutes to about 60 minutes. Generally, shorter times are used in combination with temperatures at the higher end of the given ranges, and longer times are used in combination with temperatures at the lower end of the given ranges. For example, a specific passage time of 1 hour can be used with a temperature of 60° C. in the heated zone to cause death of Pseudomonas aeruginosa cells.

The heat source useful in the current method can be any electrical resistance heater in direct contact with the heated zone, or a heat exchanger in direct contact with the heated zone and supplied with heated air or heated fluid, or a microwave heating device with the heated zone in its radiation field, magnetic induction or direct heating by passing electrical current down a tubing of suitable metal can also be used. Electrical resistance heating tape is particularly useful. The presence of such heated zone will prevent back growth of any cells that might have entered from the bioreactor back into the feed line and consequently prevent potential contamination of the feed reservoir.

### Growth Medium

Growth medium useful in the present invention includes at least one growth substrate (compounds that supply mass and energy for cell growth); and may include electron acceptors; nitrogen and phosphorus sources as well as various trace elements such as vitamins and metals that are usually required, in addition to growth substrate and nitrogen sources, for cell growth and activity.

Nutritional components of any growth medium are components that may include the following substances, alone or in combination: one or more carbon source, added at greater than 20 ppm; one or more electron acceptor for microbial cell growth (for anaerobic growth conditions), added at greater than 50 ppm; a source of nitrogen, added at greater than 1 ppm; a source of phosphorus, added at greater than 1 ppm; a source of trace nutrients, such as vitamins and metals, added at greater than 1 ppm.

Useful nutritional components contemplated herein for the growth medium include those containing at least one of the following elements: C, H, O, P, N, S, Mg, Fe, or Ca. Non-limiting examples of such inorganic compounds include: PO₄²⁻, NH₄⁺, NO₂⁻, NO₃⁻, and SO₄²⁻ amongst others. In case of microbial cells, growth substrates can include sugars, organic acids, alcohols, proteins, polysaccharides, fats, hydrocarbons or other organic materials known in the art of microbiology to be subject to microbial decomposition. Nutritional components may include major nutrients containing nitrogen and phosphorus (non-limiting examples can include NaNO₃, KNO₃, NH₄NO₃, Na₂HPO₄, K₂HPO₄, NH₄Cl); vitamins (non-limiting examples may include folic acid, ascorbic acid, and riboflavin); trace elements (non-limiting examples may include B, Zn, Cu, Co, Mg, Mn, Fe, Mo, W, Ni, and Se); buffers for environmental controls; catalysts, including enzymes; and both natural and artificial electron acceptors (non-limiting examples may include SO₄²⁻, NO₃⁻², Fe⁺³, humic acid, mineral oxides, quinone compounds, CO₂, O₂, and combinations thereof).

The nutritional components in the medium should be maintained during the heat treatment in the heated zone of the feed line, so that growth of cells is supported in the bioreactor. One skilled in the art can readily determine the effect of specific passage time and temperature in the heated zone on the ability of a specific medium to support growth of cells as shown herein in Example 2. In an embodiment of the current method, the components of the SIB medium of Example 2 used for Shewanella putrefaciens LH4:18 (ATCC PTA-8822) cell growth maintained its nutritional value after it was heated to 60° C. for six hours.

### Cells Useful for the Present Invention

Cells, including microbes, mammalian cells or plant cells are useful for the current disclosure and can be used free or immobilized on inert, insoluble materials such as glass beads or calcium alginate. For example the current invention can be useful for bioreactors growing eukaryotic cells such as algae and yeasts and a variety of tissue cell lines, e.g. cell lines like hybridomas, insect, lymphocytes, HeLa cells, tobacco and soya cells.

### Microbial Cells

For the purposes of the current disclosure, any microbial cells (bacteria, fungi and yeasts), amenable to growth in bioreactors, that may be Gram positive or Gram negative, and comprising classes of strict aerobes, facultative aerobes, obligate anaerobes, and denitrifiers can be used. The bioreactor can comprise only one particular microbial species or can comprise two or more species of the same genera or a combination of different genera of microbes, including with one or more species of each.

Examples of microbial cells useful for the disclosed method include, but not limited to: Comamonas, Fusibacter, Marinobacterium, Petrotoga, Shewanella, Pseudomonas, Vibrio, Thauera, Microbulbife, Corynebacteria, Achromobacter, Acinetobacter, Arthrobacter, Bacilli, Nocardia, Vibrio, Actinomycetes, Escherichia, Salmonella, Arthrobacter, Acetobacter, Candida, Aspergilli, Saccharomyes, Zymomonas and Penicillium.

The present invention is particularly useful when cells grown in a bioreactor have properties promoting contamination of feeding medium stored in a connected reservoir. Properties of cells including motility and formation of biofilms may promote back growth into feeding lines and medium reservoirs connected to a bioreactor in which the cells are grown. In one embodiment of the disclosed method, Pseudomonas aeruginosa cells can be used in the bioreactor. In another embodiment Shewanella putrefaciens LH4:18 (ATCC PTA-8822) cells can be used in the bioreactor. In another embodiment Pseudomonas stutzeri LH4:15 (ATCC PTA-8823) cells can be used in the bioreactor. In another embodiment Pseudomonas stutzeri strain BR5311 (ATCC PTA-11283) cells can be used in the bioreactor. In another embodiment Arcobacter sp. strain 97AE3-12 (ATCC PTA-11409) or strain 97A E3-3 (ATCC PTA-11410) cells can be used in the bioreactor. In another embodiment Thauera aromatica (ATCC PTA-9497) cells can be used to in the bioreactor

Techniques and various suitable growth media for growth and maintenance of aerobic and anaerobic microbial cells are well known in the art and have been described in “Manual of Industrial Microbiology and Biotechnology” (A. L. Demain and N. A. Solomon, ASM Press, Washington, D.C., 1986) and “Isolation of Biotechnological Organisms from Nature”, (Labeda, D. P. ed. p 117-140, McGraw-Hill Publishers,1990).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and make various changes and modifications to the invention to adapt it to various uses and conditions.

### General Methods

Optical density was measured using a Beckman Coulter DU 7500 with a 1 centimeter path length cell as is well known in the art.

Miller's Lauria Bertani (LB) growth medium was purchased from Mediatech, Manassas, Va.

### Example 1

### Heat Kill of Pseudomonas Aeruginosa Cells

In this Example, an inoculum (0.5 milliliter, ml) of frozen cells of Pseudomonas aeruginosa were aerobically grown overnight in 25 ml of the LB growth medium in a 250 ml capacity shake flask at room temperature with shaking (200 revolution per minute, rpm). Following overnight growth, cells were transferred to 20 ml-capacity capped serum vials which contained 10 ml of the LB growth medium plus 2 grams per liter (g/l) of NaNO₃. Initial optical density of the cell suspension at 600 nanometer (OD₆₀₀ nm), in duplicate serum vials, was approximately 0.0001, equivalent to about 10⁵ cells per milliliter (cells/ml). The cell suspensions in these vials were exposed to the following treatments: (1) no heat—control, (2) 0.5 hours (h) in a 60° C. water bath, (3) 1.5 h in a 60° C. water bath, and (4) 2.5 h in a 60° C. water bath. The growth medium took approximately 0.5 h to reach the temperature of 60° C. upon immersion in the 60° C. water bath. Following heat treatment, vials were immediately chilled in cold water/ice slush. Once cooled, the vials were incubated for 10 days at 30° C. After 10 days vials were checked for turbidity (growth), which indicated survival of some of the cells through the heat treatment of the vial. During the 10 days incubation the surviving cells had consumed the growth medium and the vials became turbid. Table 1 summarizes the results obtained following one h or more heat treatment at 60° C. which resulted in complete kill of the Pseudomonas aeruginosa cell suspension.

TABLE 1
Exposure time at 80° C. temperature
needed to kill Pseudomonas aeruginosa

| | | | Growth | Complete kill |
|---|---|---|---|---|
| | | Time | positive, | of 10⁵ |
| | Time with | exposed to | replicate | cells/ml |
| Treatment | heating, h | 60° C., h | results | suspension |
| 1 | 0 | 0 | +, + | No |
| 2 | 0.5 | 0 | +, + | No |
| 3 | 1.5 | 1 | −, − | Yes |
| 4 | 2.5 | 2 | −, − | Yes |

### Example 2

### Determining Temperature Compatibility of the Growth Medium

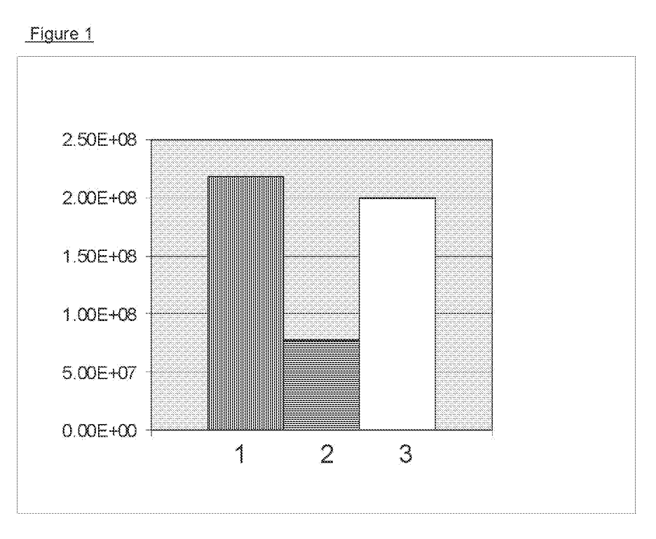

Some growth media may contain components that are heat sensitive. In such cases one option is to find a heating regime that allows substantially retaining the nutritional value of the nutritional components of the medium, while still killing microbial cells that can potentially contaminate the feed line and the feed reservoir by growing back from the bioreactor. Shewanella putrefaciens (LH4:18—ATCC PTA-8822) cells were grown in the SIB medium containing the following composition: per 1 liter of di-ionized water: NaHCO₃, 0.138 g; CaCl₂*6H₂O, 0.39 g; MgCl₂*6H₂O, 0.220 g: KCl, 0.090 g; NaCl, 11.60 g; Trace metals [a solution of 25% HCl, 10 ml/l; FeCl₂*4 H₂O, 1.50 g/l; ZnCl₂, 70 milligrams per liter (mg/l); MnCl₂*4 H₂O, 100 mg/l; H₃BO₃, 6 mg/l; CoCl₂*6 H₂O, 190 mg/l; CuCl₂*2 H₂O, 2 mg/; NiCl₂*6 H₂O, 24 mg/l; Na₂MoO₄*2 H₂O, 36 mg/l]; 1 ml, Vitamins [a solution of Vitamin B12, 100 mg/l; p-Aminobenzoic acid, 80 mg/l; D(+)-Biotin, 20 mg/l; Nicotinic acid, 200 mg/l; Calcium pantothenate, 100 mg/l; Pyridoxine hydrochloride, 300 mg/l; Thiamine-HCl*2 H₂O, 200 mg/l; Alpha-lipoic acid, 50 mg/l]; 1 ml, Na₂HPO₄, 0.015 g; NH₄Cl, 0.029 g; Na-Lactate, 0.124 g; Na-Fumarate, 0.422 g. This medium was screened for its temperature sensitivity when exposed to 60° C. for different periods of time. Four replicate vials containing the medium were used for each time period tested. Following heat treatment and after cooling, the medium was inoculated with approximately 10⁵ cells/ml of Shewanella putrefaciens. The test vials were then incubated at 16° C. for six days. At the end of the incubation, cell counts were determined by streaking a solution from the vials on the LB agar plates. Comparing the number of the cells/ml following their growth on the LB agar plates in the control vials that were not heated with the number of the cells/ml in the test samples that were heated (FIG. 1), showed that the medium substantially retained the nutritional value of its nutritional components after six hours of heating, since the number of the cells/ml in this test was about equal to the control that was not heated. In contrast heating for 12 hours caused loss in nutritional value and resulted in reduced cell yield from about 2×10⁸ cells/ml to about 0.6×10⁸ cells/ml. Additional testing, similar to that described in Example 1, showed that Shewanella putrefaciens was killed by one h exposure to 60° C. In summary, these results underlined that while heat treatment for one h at 60° C. killed the Shewanella putrefaciens cells, it did not adversely affect the medium and allowed it to substantially retain the nutritional value of its nutritional components.

### Example 3

### Maintenance of Sterility of Feed Line and Feed Reservoir in a Bioreactor System in a Drill Core of an Oil Reservoir

A bioreactor system was set up with a drill core sample from an oil reservoir geological formation. The bioreactor consisted of the drill core, consisting of porous rock, encased in a metal housing that operated under approximately 1200 pressure per square inch (psi) (8273.7 kilopascal) which was a core flood apparatus (Core Laboratories, Houston, Tex.). The feed reservoir consisted of a metal cylinder with a tight fitting metal plunger to deliver the growth medium to the bioreactor. Sterile growth medium was pumped from the feed reservoir into the bioreactor through a metal tube, serving as the feed line, which was connected to the feed reservoir. The growth medium, described in Example 2, supported growth of Shewanella putrefaciens LH4:18 (ATCC PTA-8822) cells under anaerobic conditions in the bioreactor. Growth medium was pumped into the bioreactor at a rate of about 1.7 milliliter per hour (ml/h) for 36 days. The feed line was heated using electrical resistance heating tape to approximately 60° C. in a section of tubing extending approximately 30 centimeters upstream of the entry into the bioreactor. The volume of the growth medium, approximately 0.8 ml, in the heated zone was sufficient to allow approximately 1 h of exposure to 60° C. as the medium passed through the feed line.

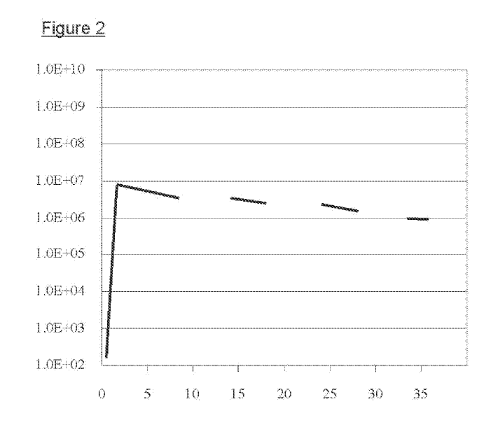

The bioreactor was inoculated on day zero with an inoculum of Shewanella putrefaciens, prepared as outlined in Example 2. Cell counts in the bioreactor, as measured by dilution plating on LB agar plates, rose rapidly to a range of about 10⁶-10⁷ cells/ml within two days (FIG. 2), and periodic testing showed that this population was maintained over the course of 36 days. Shewanella putrefaciens is a motile organism and forms biofilm. Either of these two mechanisms, i.e., motility or biofilm formation, would be expected to allow back growth up the feed line and into the feed reservoir over the extended period of the bioreactor operation. However, further testing showed no detectable contamination in the medium contained in the feed reservoir after 37 days, as evidenced by no growth when the medium was plated on an agar plate. The walls of the feed reservoir were clean, i.e. no biofilm was observed on these walls since the heated zone in the feed line had killed any cells entering that zone. Thus cells growing in the bioreactor were prevented from back growth in the feed line which could have resulted in contaminating the feed reservoir. If the feed reservoir had become contaminated, the contaminating cells would have rapidly consumed the nutrients in the medium, thus removing the nutritional components required for growth of the cells in the bioreactor resulting in their growth and/or metabolism failure. The heated zone in the feed line is therefore effective at maintaining feed reservoir's sterility for the extended operation.

What is claimed is:

1. A method for preventing contamination in a bioreactor system comprising applying heat to a feed line supplying a growth medium from a feed reservoir to a bioreactor via the feed line, said heat applied to establish a heated zone in the feed line; wherein said heated zone is sufficient to prevent contamination of the feed line or the feed reservoir with cells from the bioreactor in the absence of any intervening physical device.

2. The method of claim 1 wherein the heated zone is established using electrical, microwave or magnetically generated heat.

3. The method of claim 1 wherein the heated zone in the feed line is established from about 0.1 centimeter to about 100 centimeters from where the feed line joins the bioreactor.

4. The method of claim 3 wherein temperature in the heated zone is from about 30° C. to about 200° C. and specific passage time is for about 0.5 minutes to about 240 minutes.

5. The method of claim 3 wherein the temperature in the heated zone is from about 40° C. to about 121° C. and specific passage time for about 0.5 minutes to about 60 minutes.

6. The method of claim 1 wherein nutritional components of the growth medium substantially retain their nutritional value.

7. The method of claim 6 wherein combination of specific passage time through the feed line in the heated zone and temperature of the feed line in the heated zone is such that during passage through the heated zone, cell death is complete and nutritional value of the growth medium is substantially retained.

8. The method of claim 1 wherein the cells in the bioreactor are cells having properties of motility, biofilm formation, or both motility and biofilm formation.

9. The method of claim 1 wherein the cells used in the bioreactor are Pseudomonas aeruginosa.

10. The method of claim 1 wherein the cells used in the bioreactor are Shewanella putrefaciens LH4:18 (ATCC PTA-8822).

11. The method of claim 1 wherein the cells used in the bioreactor are Pseudomonas stutzeri LH4:15 (ATCC PTA-8823).

12. The method of claim 1 wherein the cells used in the bioreactor are Arcobacter sp. 97AE3-12 (ATCC PTA-11409) or Arcobacter sp. 97AE3-3 (ATCC PTA-11410).

13. The method of claim 1 wherein the bioreactor comprises a drill core sample from an oil reservoir.

14. A bioreactor system comprising at least one reservoir containing a growth medium, a bioreactor, and at least one feed line connecting the reservoir and bioreactor having a heat source attached to at least a portion of the feed line that is a heated zone.

15. The method of claim 14 wherein the bioreactor comprises a drill core sample from an oil reservoir.
